(19) United States
(12) Patent Application Publication
(10) Pub. No.: US 2023/0146083 A1
(43) Pub. Date: May 11, 2023

(54) RISK-BASED ALGORITHMIC SYSTEM FOR FITTING A CONDOM OR A PROPHYLACTIC BASED ON GIRTH MEASUREMENT

(71) Applicant: Neville Kudzaishe Muringayi, Coral Gables, FL (US)

(72) Inventor: Neville Kudzaishe Muringayi, Coral Gables, FL (US)

(21) Appl. No.: 17/839,403

(22) Filed: Jun. 13, 2022

Related U.S. Application Data
(60) Provisional application No. 63/263,772, filed on Nov. 9, 2021.

(51) Int. Cl. G06Q 30/06

(52) U.S. Cl. CPC G06Q 30/0631; G06Q 30/0635

(57) ABSTRACT

A sizing method and guide, providing condom or prophylactic fitting and sizing based on a girth measurement. The method teaches an algorithm and method for creating a sizing chart to only use penis girth to correlate it to a condom size. Condom circumference is the first variable that is determined in the algorithm. The second variable, “alpha” (a), is known as the risk variable. The third variable is penis girth. A range of penis girths with an alpha that is between at least 0.81 and at most 0.88 for a given condom nominal width mitigates the risk of condom failure. The algorithm creates a sizing chart only using penis girth to correlate it to a condom nominal width and a tolerance factor to consider when choosing a condom nominal width size based on penis girth.

### FEDERALLY SPONSORED RESEARCH

Not Applicable

### SEQUENCE LISTING OR PROGRAM

Not Applicable

### TECHNICAL FIELD OF THE INVENTION

The present invention relates generally to a method for condom or prophylactic fitting. More specifically, the present invention relates to providing condom or prophylactic fitting and sizing based on girth using a risk-based algorithm.

### BACKGROUND OF THE INVENTION

Seventy percent of men use the wrong condom size, and a poorly fitted condom can frustrate its purpose through facilitating slippage and breakage. Research has shown that there is a statistically significant correlation between penile girth and condom breakage and comfort. Pursuant to the research girth determinative factor in properly sizing a condom, yet currently there is no way to size a condom using only girth. US Patent Publication 2014/0161328 to Ireland, et al. discloses a system and method of selecting a prophylactic for a body part, such as a condom for a penis. The system is run as a software application running on a handheld electronic device that contains an integrated digital camera. To operate the software application, a user selects a measurement standard having known dimensions. The measurement standard is positioned near the body part and a digital image is taken. Points along the periphery of the imaged body part are manually selected. From the selected points, the software application can calculate the distance between the points and size the body part. In the case of a penis, the length and girth of the penis can be readily calculated by the software application. Once the body part is sized, the software application uses the measured sizes to determine what prophylactic device would fit best. This information is communicated to the user.

US Patent Publication 2006/0032506 to Sadlo teaches an apparatus and method are provided to adapt word processing software and printers to enable a prophylactic sizing kit to be delivered via the Internet to a remote site, to correlate penile measurements with prophylactic size to make or order, and to remit correlated order information to a point of sale.

US Patent Publication 2012/0191013 teaches a method for fitting a wearable covering for a body part comprises the steps of running a program on a portable terminal; determining with the portable terminal specific geometrical points regarding the body part; using the determined specific geometrical points to determine an approximate shape of the body part; comparing the determined approximate shape with a table of different sized wearable coverings for the body part; and selecting a size for the wearable covering.

In short, the problem with other prior art and current market devices is that they either utilize complex measuring systems or ask for unnecessary dimensions—i.e., they do not allow the user to understand what measurement is decisive for condom usage, thereby diminishing the importance in the mind of the user as the superfluous dimension of length is focused on by the user.

Specifically, existing solutions for correlating penile measurements to a corresponding condom's size are inefficient for three reasons. The first reason is that they require the user to measure both their length and their girth. The length of a penis is not a significant determinate factor in the safety or comfort of a corresponding condom size as demonstrated in a study published in the National Institute of Health. The second reason is that current solutions require the user to use a brand-new measuring system to measure their penile length and girth. The third and last reason is that current solutions require the user to print out the size guide to match their penile measurements to a corresponding condom size, introducing an extra step that does not allow for efficient input of data from the user to quickly find a corresponding condom size.

As can be seen, there is a need for a risk-based algorithmic system of fitting a condom or a prophylactic based on girth, wherein the system is embodied in an apparatus and method to correlate only penile girth to one or multiple corresponding prophylactic sizes to make or order and to remit the correlated order information to a point of sale.

### Definitions

Unless stated to the contrary, for the purposes of the present disclosure, the following terms shall have the following definitions:

“Application software” is a set of one or more programs designed to carry out operations for a specific application. Application software cannot run on itself but is dependent on system software to execute. Examples of application software include MS Word, MS Excel, a console game, a library management system, a spreadsheet system etc. The term is used to distinguish such software from another type of computer program referred to as system software, which manages and integrates a computer's capabilities but does not directly perform tasks that benefit the user. The system software serves the application, which in turn serves the user.

The term “app” is a shortening of the term “application software”. It has become very popular and in 2010 was listed as “Word of the Year” by the American Dialect Society “Apps” are usually available through application distribution platforms, which began appearing in 2008 and are typically operated by the owner of the mobile operating system. Some apps are free, while others must be bought. Usually, they are downloaded from the platform to a target device, but sometimes they can be downloaded to laptops or desktop computers.

“API” In computer programming, an application programming interface (API) is a set of routines, protocols, and tools for building software applications. An API expresses a software component in terms of its operations, inputs, outputs, and underlying types. An API defines functionalities that are independent of their respective implementations, which allows definitions and implementations to vary without compromising each other.

“GUI”. In computing, a graphical user interface (GUI) sometimes pronounced “gooey” (or “gee-you-eye”)) is a type of interface that allows users to interact with electronic devices through graphical icons and visual indicators such as secondary notation, as opposed to text-based interfaces, typed command labels or text navigation. GUIs were introduced in reaction to the perceived steep learning curve of command-line interfaces (CLIs), which require commands to be typed on the keyboard.

A “mobile app” is a computer program designed to run on smartphones, tablet computers and other mobile devices, which the Applicant/Inventor refers to generically as “a computing device”, which is not intended to be all inclusive of all computers and mobile devices that are capable of executing software applications.

A “mobile device” is a generic term used to refer to a variety of devices that allow people to access data and information from where ever they are. This includes cell phones and other portable devices such as, but not limited to, PDAs, Pads, smartphones, and laptop computers.

A “module” in software is a part of a program. Programs are composed of one or more independently developed modules that are not combined until the program is linked. A single module can contain one or several routines or steps.

A “module” in hardware, is a self-contained component.

A “software application” is a program or group of programs designed for end users. Application software can be divided into two general classes: systems software and applications software. Systems software consists of low-level programs that interact with the computer at a very basic level. This includes operating systems, compilers, and utilities for managing computer resources. In contrast, applications software (also called end-user programs) includes database programs, word processors, and spreadsheets. Figuratively speaking, applications software sits on top of systems software because it is unable to run without the operating system and system utilities.

A “software module” is a file that contains instructions. “Module” implies a single executable file that is only a part of the application, such as a DLL. When referring to an entire program, the terms “application” and “software program” are typically used. A software module is defined as a series of process steps stored in an electronic memory of an electronic device and executed by the processor of an electronic device such as a computer, pad, smart phone, or other equivalent device known in the prior art.

A “software application module” is a program or group of programs designed for end users that contains one or more files that contains instructions to be executed by a computer or other equivalent device.

A “computer system” or “system” consists of hardware components that have been carefully chosen so that they work well together and software components or programs that run in the computer. The main software component is itself an operating system that manages and provides services to other programs that can be run in the computer. The complete computer made up of the CPU, memory and related electronics (main cabinet), all the peripheral devices connected to it and its operating system. Computer systems fall into two categories: clients and servers.

A “User” is any person registered to use the computer system executing the method of the present invention.

A “web application” or “web app” is any application software that runs in a web browser and is created in a browser-supported programming language (such as the combination of JavaScript, HTML and CSS) and relies on a web browser to render the application.

A “website”, also written as Web site, web site, or simply site, is a collection of related web pages containing images, videos or other digital assets. A website is hosted on at least one web server, accessible via a network such as the Internet or a private local area network through an Internet address known as a Uniform Resource Locator (URL). All publicly accessible websites collectively constitute the World Wide Web.

A “web page”, also written as webpage is a document, typically written in plain text interspersed with formatting instructions of Hypertext Markup Language (HTML, XHTML). A web page may incorporate elements from other websites with suitable markup anchors.

Web pages are accessed and transported with the Hypertext Transfer Protocol (HTTP), which may optionally employ encryption (HTTP Secure, HTTPS) to provide security and privacy for the user of the web page content. The user's application, often a web browser displayed on a computer, renders the page content according to its HTML markup instructions onto a display terminal. The pages of a website can usually be accessed from a simple Uniform

Resource Locator (URL) called the homepage. The URLs of the pages organize them into a hierarchy, although hyperlinking between them conveys the reader's perceived site structure and guides the reader's navigation of the site.

### SUMMARY OF THE INVENTION

The present invention takes only the user's girth into account and converts that to a corresponding prophylactic size based on the risk of slippage and breakage to the user, while other guides need require the user to measure both girth and length to recommend a condom size and rely on a printable size guide to do so.

The present invention measures the only dimension that actually matters for condom sizing, girth, the measurement is obtain through an intuitive means, assesses the level of risk for slippage and breakage relative to the user's penis measurements, and converted to a condom size that properly fits.

The present invention is a sizing method and guide, which can be presented to a user in a physical embodiment or in an electronic embodiment through the use of a computer or mobile electronic device running and executing an application or other system software providing condom or prophylactic fitting and sizing based on girth measurement. The present invention may include a physical or electronic representation of the tabular size chart.

The size guide/chart helps users more accurately choose a condom size based on their penile girth. The size guide/chart correlates a range of girths to an appropriate condom size. These condom size to girth correlations are an estimate that are meant to guide the user to a better fitting condom size.

In order to obtain a girth measurement, a user simply prints out the size guide ruler page or uses a tailor's tape (soft measuring tape), measures the girth of their erect penis, and correlates that girth to an appropriate condom size.

In an alternative, the electronic system of the present invention can display or provide for download. In this embodiment of the present invention, a size chart correlating a series of prophylactic sizes to their corresponding range of penile girths may be electronically provided. A user interface displaying the series of prophylactic sizes to their corresponding range of penile girths would need to be designed and made accessible via a computer or portable terminal to the user. A point-of-sale system allowing the user to make or order and to remit the correlated order information to a point of sale using aforementioned computer or portable terminal is contemplated.

The present invention, in its novel and non-obvious method and application teaches an algorithm and method for creating a sizing chart to only use penis girth to correlate it to a condom nominal width and a tolerance factor to consider when choosing a condom nominal width size based on penis girth.

The condom size chart of the present invention is a unique methodology that allows users to correlate their penis girth to one of the condom sizes offered for sale by the system. The basis for helping the user find the right condom size based solely on their girth is the slippage-breakage risk algorithm taught by the present invention. The end goal is to assess the level of risk a user's penis girth has when it's correlated to a particular condom nominal width and therefore recommend a condom size that will maximize safety during usage.

The condom circumference is the first variable that is determined in the algorithm. The condom circumference is measured in millimeters and is found by multiplying a condom's nominal width (provided by the manufacturer) by 2.

The second variable, “alpha” (a), is known as the risk variable. Alpha represents the risk the user is exposed to for breaking or slipping out of a condom. This risk is a ratio between the condom circumference (not nominal width) and penis girth, which should be between 80% to 90%. Through research, unexpected results where shown that more than 90% and the User is at risk of slippage, and less than 80% will put the User at a greater risk of breakage. Alpha is represented as a range of percentages converted to decimals between 0.8 to 0.9 with a 0.1 step (i.e. 0.8, 0.81, 0.82, etc.).

The third variable is penis girth. This is a measurement that the user inputs themselves and is the circumference of an erect penis around the thickest part of the shaft. This is typically measured in inches in America and millimeters everywhere else in the world.

For the sake of safety, based on the research and unexpected results, the method of the present invention teaches a range of penis girths with an alpha that is between at least 0.81 and at most 0.88 for a given condom nominal width to mitigate the risk of condom failure for the user.

### BRIEF DESCRIPTION OF THE DRAWINGS

The accompanying drawings, which are incorporated herein form a part of the specification, illustrate the present invention and, together with the description, further serve to explain the principles of the invention and to enable a person skilled in the pertinent art to make and use the invention.

FIG. 1 is a tabular size chart matching a range of penile girths to corresponding prophylactic sizes. The tabular size chart correlates a series of prophylactic sizes to their corresponding range of penile girths. The tabular size chart can be made electronically accessible to users via a user interface that is accessible via a computer or portable terminal.

FIG. 2 is a flow chart illustrating the sizing method of the present invention enabled by and presented to a user through a computer system and/or mobile device.

FIG. 3 is a simulated screen shot of a PDF print out or GUI display in one embodiment of the present invention providing a screen display or print out to provide a user with a ruler for use by the user to take a measurement.

FIG. 4 is an excel spreadsheet illustrating the application of the algorithm taught by the present invention and the output that is produced as a result of the inputs in the algorithm and represents the recommended penis girths in relation to the risk variable for a given condom nominal width.

FIG. 5 is an excel spreadsheet illustrating the lower and upper bounds of condom size with and without a tolerance factor being considered by the algorithm taught by the present invention.

### DETAILED DESCRIPTION OF THE INVENTION

In the following detailed description of the invention of exemplary embodiments of the invention, reference is made to the accompanying drawings (where like numbers represent like elements), which form a part hereof, and in which is shown by way of illustration specific exemplary embodiments in which the invention may be practiced. These embodiments are described in sufficient detail to enable those skilled in the art to practice the invention, but other embodiments may be utilized and logical, mechanical, electrical, and other changes may be made without departing from the scope of the present invention. The following detailed description is, therefore, not to be taken in a limiting sense, and the scope of the present invention is defined only by the appended claims.

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention. Referring to the figures, it is possible to see the various major elements constituting the present invention.

The present invention is a sizing method and guide, which can be present to a user in a physical embodiment or in an electronic embodiment through the use of a computer or mobile electronic device running and executing an application or other system software providing condom or prophylactic fitting and sizing based on girth measurement. The present invention may include a physical or electronic representation of the tabular size chart 100 represented in the figures.

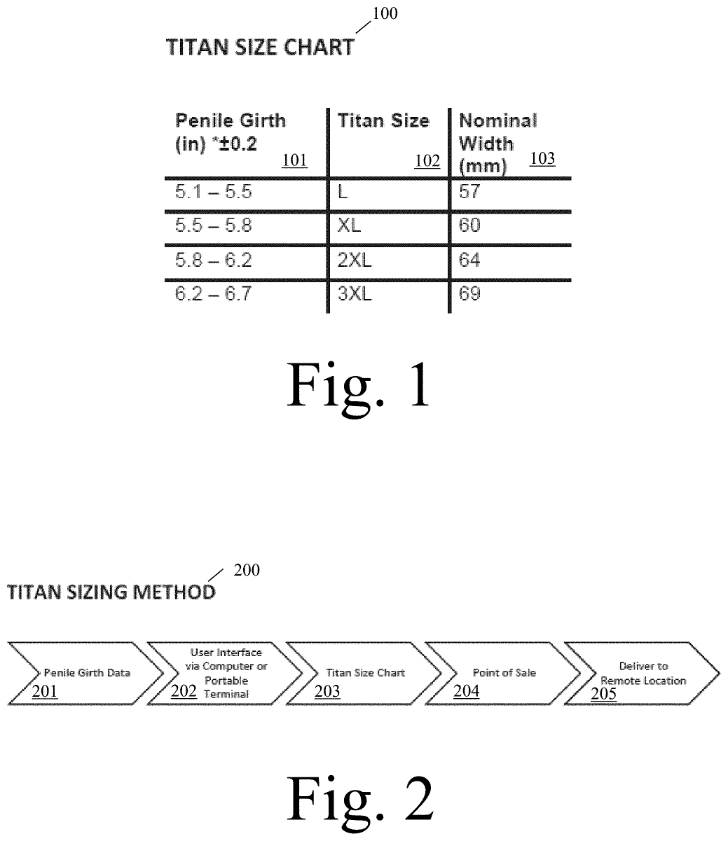

The size guide/chart 100 as shown in FIG. 1 was developed to help users more accurately choose a condom size based on their penile girth. The size guide/chart 100 correlates a range of girths to an appropriate condom size. These condom size to girth correlations are an estimate that are meant to guide the user to a better fitting condom size.

In order to obtain a girth measurement, a user simply prints out the size guide ruler page 300 or uses a tailor's tape (soft measuring tape), measures the girth of their erect penis, and correlates that girth to an appropriate condom size.

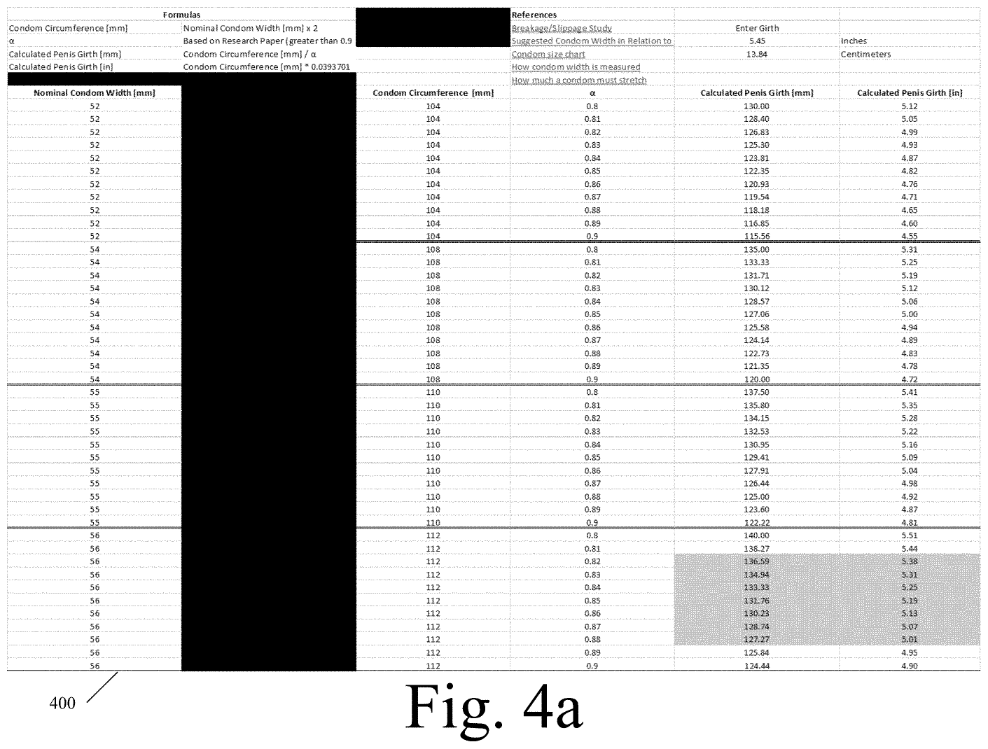

FIG. 3 is a simulated screen shot of a PDF print out or GUI display in one embodiment of the present invention providing a screen display or print out providing a size guide ruler page 300 to provide a user with a ruler for use by the user to take a measurement if a tailor's tape or other measurement means is not readily available.

The size guide ruler page 300 also provides directions and steps as taught by the method of the present invention for a user to take an accurate girth measurement to correlate, using the size chart 100 to guide the user to a better fitting condom size and making a more informed selection.

In this embodiment, the first step is to print out the size guide 301 and make sure that the size guide is printed out correctly to avoid any sizing errors. In this embodiment, a driver's license can be placed on top of a box 305 to verify the guide is printed correctly and has the proper dimensions and has not be scaled to an incorrect dimension by the printer or other hardware of software of the computer system running the software application of the present invention. If the card doesn't fit, the printed page is not correct and the user may have to adjust their printer's settling, such as turning off or adjusting the scaling to verify or adjust printing at 100% size.

Step two is to cut out the ruler 302. Step three is to measure girth 303. When measuring girth, a user should make sure their penis is fully erect before measuring. The user should then gently wrap the ruler 306 around the thickest part of the penis shaft, noting where the ends of the ruler meet as that is the girth measurement number.

Finally, in the fourth step, the user should find their size 304 by matching the corresponding girth with the appropriate condom size in the size table 100. Through rigorous testing, the Inventor has found that there is a girth tolerance that needs to be considered. A girth tolerance of +/−0.2″ is what has been specified . In other words, for the user to find their size they match the corresponding girth with the appropriate condom size/systemic suggestion from the tabular size chart. The systemic suggestions account for tolerances. For example, if a user's girth is 5.5 inches, the tolerance would be 5.3″ to 5.7″, which corresponds to a size suggestion.

In an alternative embodiment shown in FIG. 3, the electronic system of the present invention can display or provide for download. In this embodiment of the present invention, a size chart 100 correlating a series of prophylactic sizes to their corresponding range of penile girths may be electronically provided. A user interface displaying the series of prophylactic sizes to their corresponding range of penile girths would need to be designed and made accessible via a computer or portable terminal to the user. A point-of-sale system allowing the user to make or order and to remit the correlated order information to a point of sale using aforementioned computer or portable terminal is contemplated.

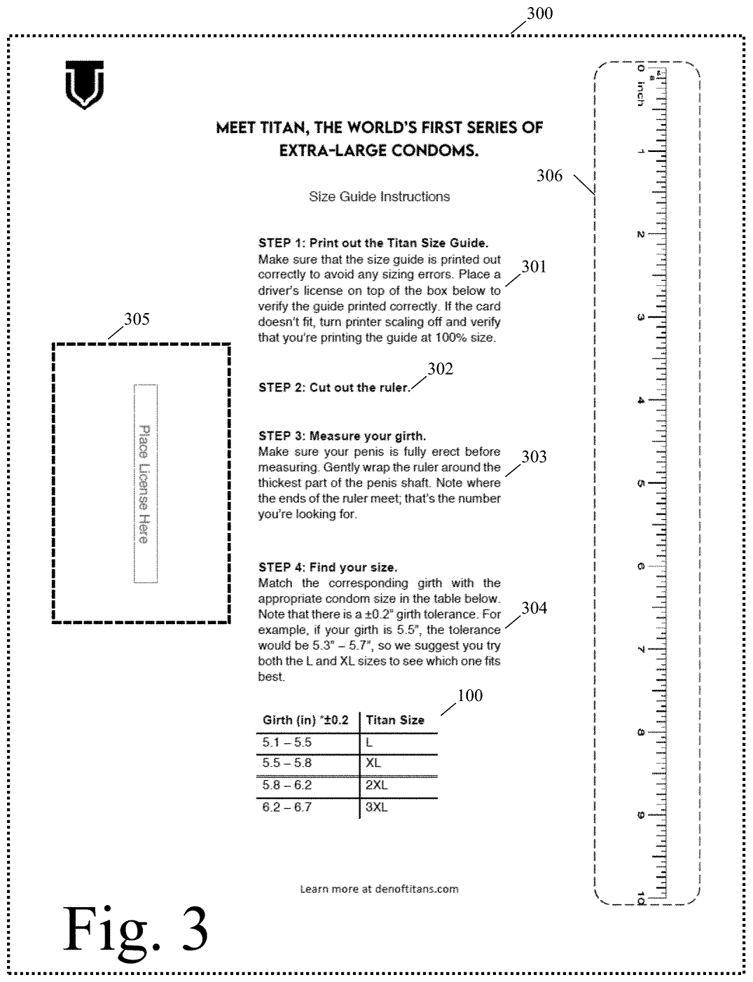

Now referring to FIG. 2, the sizing method 200 for an electronic embodiment of the present invention is illustrated in by the process method flow chart providing the method steps enabled by the software application running on a computer hardware platform of the present invention, which contains penile girth data 201.

The first step for the user is to access the user interface using a computer or portable terminal 202. From the terminal, the User proceeds to take their pre-measured penile girth and correlate it to one or multiple prophylactic sizes on a size chart 203 to make or order and remit the correlated order information to the point of sale 204. Finally, the system will process the point of sale order and deliver the ordered product to a remote location 205.

In this embodiment, the user can access the size guide 100 through a website or mobile application or an app running on a computer platform. The software application executing the method and process of the present invention in electronic form will be available for access via a computer or mobile phone interface.

The electronically delivered size guide of the present invention is similar to prior art devices due to the similarities in the method of determining a user's condom size. Both the size guide and some prior art devices require the user to double check their printer's scaling is accurate when the sheets of paper are printed out, both provide instructions for use, both provide an accurately sized measuring tool that can be cut out by the user, and they take penile measurements into account to determine the right size condom to choose.

Where the present invention improves on the prior art in a novel and non-obvious manner is that the present invention uses an algorithm to provide a size correlation. Known prior art devices ask the user to measure both penile length and penile girth, whereas the size guide of the present invention only asks for penile girth. Other prior art devices also teach a complex and proprietary system of measurement using a combination of letters and numbers to measure a user's penis. The size guide of the present invention uses a standard metric/imperial system ruler. Known prior art devices do not provide the condom-size to penis measurement correlations on the sheet of paper. The size guide of the present invention does provide the condom-size to penile girth correlations on the sheet of paper.

The condom size chart of the present invention is a unique methodology that allows users to correlate their penis girth to one of the condom sizes offered for sale by the system. The basis for helping the user find the right condom size based solely on their girth is the slippage-breakage risk algorithm taught by the present invention. The end goal is to assess the level of risk a user's penis girth has when it's correlated to a particular condom nominal width and therefore recommend a condom size that will maximize safety during usage.

The condom circumference is the first variable that is determined in the algorithm. The condom circumference is measured in millimeters and is found by multiplying a condom's nominal width (provided by the manufacturer) by 2.

The second variable, “alpha” (a), is known as the risk variable. Alpha represents the risk the user is exposed to for breaking or slipping out of a condom. This risk is a ratio between the condom circumference (not nominal width) and penis girth, which should be between 80% to 90%. Through research, unexpected results where shown that more than 90% and the User is at risk of slippage, and less than 80% will put the User at a greater risk of breakage. Alpha is represented as a range of percentages converted to decimals between 0.8 to 0.9 with a 0.1 step (i.e. 0.8, 0.81, 0.82, etc.).

The third variable is penis girth. This is a measurement that the user inputs themselves and is the circumference of an erect penis around the thickest part of the shaft. This is typically measured in inches in America and millimeters everywhere else in the world.

TABLE 1
Breakage Risk Algorithm Formulas
Breakage and Slippage Risk Algorithm Formulas

Condom Circumference [mm] = Nominal Width [mm] × 2
* Assuming penis girth is measured in millimeters
Suggested Nominal Width [mm] = (Penis Girth [mm] × α) ÷ 2
Suggest Penis Girth [mm] = Condom Circumference [mm] ÷ α
* Assuming penis girth is measured in inches
Suggested Nominal Width [mm] = (Penis Girth [in] × 25.4 × α) ÷ 2
Suggest Penis Girth [in] = (Condom Circumference [mm] × 0.039370) ÷ α

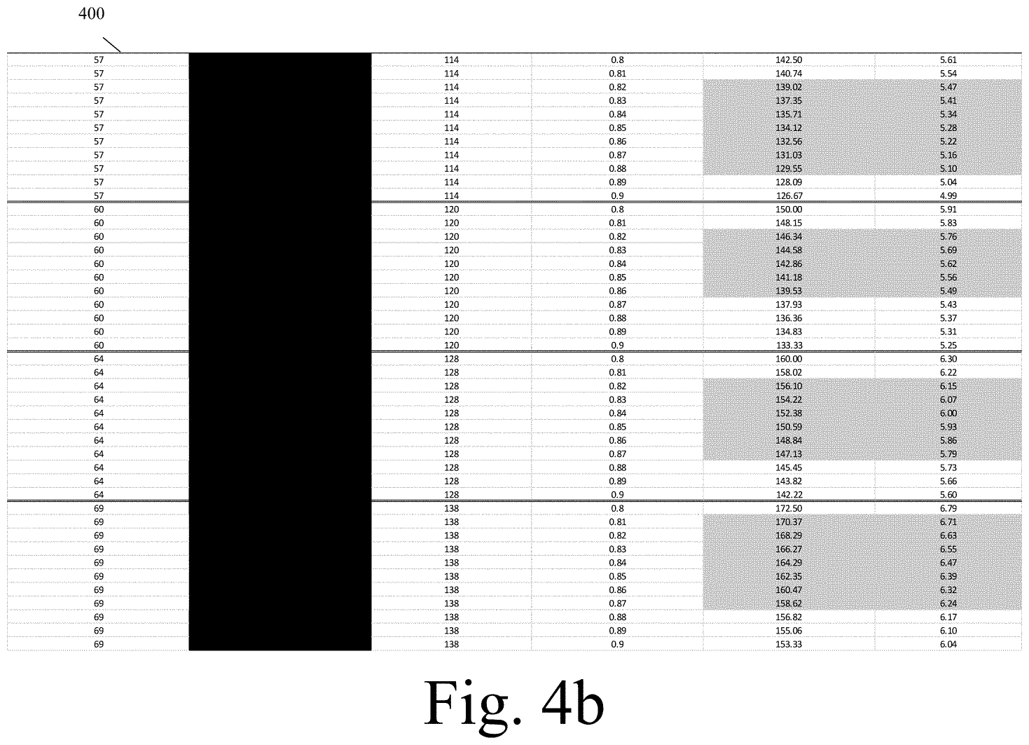

Now referring to FIGS. 4a and 4b, a size chart 400 is produced as a result of the inputs in the algorithm and represents the recommended penis girths in relation to the risk variable for a given condom nominal width.

For the sake of safety, based on the research and unexpected results, the method of the present invention teaches a range of penis girths with an alpha that is between at least 0.81 and at most 0.88 for a given condom nominal width to mitigate the risk of condom failure for the user.

Although the size chart 400 represents the risk associated between a given penis girth and condom nominal width, there is a bit of overlap.

For example, a penis girth of 5.43 inches has an alpha of 0.87 for a condom nominal width of 60 mm. That same penis girth, 5.43 inches, has an alpha of 0.83 for a condom nominal width of 57 mm. This means that a penis girth of 5.43 inches is more likely to slip when using a 60 mm condom. Because of this overlap, the method of the present invention uses an alpha range between 0.82 and 0.86 for a condom with a nominal width of 60 mm to suggest a range of penis girths. This cut-off range is represented with grey-colored cells in the Size Chart.

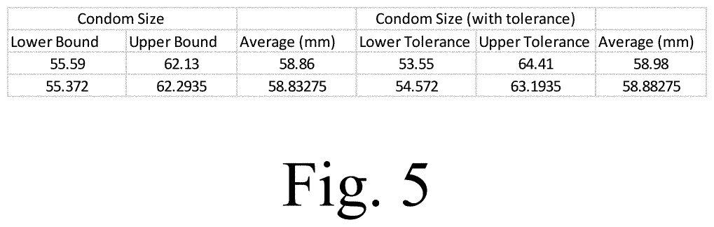

Now referring to FIG. 5, there is a ±0.2 inch penis girth tolerance as a result of the sizing and risk overlap mentioned above.

For example, if a user's girth is 5.7 inches, the tolerance would be between 5.5 inches and 5.9 inches, so a user would be recommended to try both the 60 mm and 64 mm nominal width condom sizes to see which one fits best.

The U.S. Centers for Disease Control and Prevention has recommended the male latex condom as the best physical barrier for preventing HIV and sexually transmitted infection (STI) transmission. Condoms are a safe and effective form of birth control that has 98% effectiveness in protecting against STIs and pregnancy. However, evidence shows that if a condom does not fit properly, its impact can reduce to 85% or less, as it may slip off during sex or during the removal of the penis from the vagina or break during intercourse. Condom error (such as incorrect application or removal of condoms) and failure (such as breakage and slippage) are common.

Rates of condom failure (breakage and slippage) vary broadly across studies. It has been estimated that condom failure ranges from 1% to 33% of a lifetime experience with condoms. In one study, of the 3,658 condoms used by 184 men, 49 (1.34%) broke and 73 (2.05%) slipped. 30 men (16.3%) experienced at least one instance of breakage and 35 (19.0%) experienced complete slippage. The author reported that the condom breakage was strongly associated with the penile circumference (width). Each additional centimeter of penile circumference increased the risk of condom breakage by 50-100%.

Condoms need to fit correctly to protect against fluids and exposed skin which could lead to STD contraction or pregnancy. To choose the right size, the girth (the width of an erect penis) is the most important factor, not length. However, most condom companies do not list their nominal width, let alone base their sizes on girth.

In order to validate the algorithm taught by the present invention and size chart, the Inventor conducted a clinical study with human participants. The primary objective of the study was to assess clinical slippage and breakage that is needed to support the performance of the 64 mm and 69 mm nominal width condoms. A total of 60 couples were recruited to participate in the study and were asked to use the Size Guide to be determined eligible for the study as well as assigned the proper condom size. Each couple was given three condoms of their respective size and given two weeks to use all of them (condom uses) and fill out a survey documenting whether a clinical slippage or breakage occurred.

The final sample included a total of 119 condom use surveys. There were 54 condom uses of condoms with nominal width dimensions of 64 mm. There were 65 condom uses of condoms with nominal width dimensions of 69 mm. There was 1 clinical breakage during vaginal intercourse of the condoms with nominal width dimensions of 69 mm, 1 clinical slippage during vaginal intercourse of the condoms with nominal width dimensions of 69mm, and 1 slippage during withdrawal of the condoms with nominal width dimensions of 69 mm. The slippage during withdrawal occurred as a result of user error since the user reported that they had failed to hold onto the condom at the base of the penis during withdrawal.

Based on the results of this study, the clinical breakage rate of the condoms with nominal width dimensions of 64 mm (54 condom uses) and 69 mm (65 condom uses)) is 0.84% (1/119). The clinical slippage rate of the Condoms(64 mm and 69 mm) is 0.84% (1/119). The total clinical failure rate is 1.68% (2/119). The condoms met the predefined total clinical failure endpoint of <5%.

Prior art size charts such as the TROJAN CONDOMS size chart lists a given TROJAN CONDOM SKU with its corresponding condom length, condom nominal width, and condom mid-body width. A recommendation for girth (single girth or range) to a given condom's nominal width in not provided in prior art size charts and that of the most popular, the TROJAN brand. These prior art size charts also do not teach, include, or in any manner suggest a tolerance factor to consider when choosing a condom nominal width size based on penis girth.

Prior art devices also commonly teach a printable size guide that allows the user to measure their penis length and girth to correlate it to an arbitrary number/letter combination to determine the right condom size. These prior art size charts also do not teach, include, or in any manner suggest a method to only use penis girth to correlate it to a condom nominal width. These prior art size charts also do not teach, include, or in any manner suggest a tolerance factor to consider when choosing a condom nominal width size based on penis girth.

The present invention, in its novel and non-obvious method and application teaches an algorithm and method for creating a sizing chart to only use penis girth to correlate it to a condom nominal width and a tolerance factor to consider when choosing a condom nominal width size based on penis girth.

The system is set to run on a computing device or mobile electronic device. A computing device or mobile electronic device on which the present invention can run would be comprised of a CPU, Hard Disk Drive, Keyboard, Monitor, CPU Main Memory, and a portion of main memory where the system resides and executes. Any general-purpose computer, smartphone, or other mobile electronic device with an appropriate amount of storage space is suitable for this purpose. Computer and mobile electronic devices like these are well known in the art and are not pertinent to the invention. The system can also be written in a number of different languages and run on a number of different operating systems and platforms.

Although the present invention has been described in considerable detail with reference to certain preferred versions thereof, other versions are possible. Therefore, the point and scope of the appended claims should not be limited to the description of the preferred versions contained herein.

As to a further discussion of the manner of usage and operation of the present invention, the same should be apparent from the above description. Accordingly, no further discussion relating to the manner of usage and operation will be provided.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

The embodiments of the invention in which an exclusive property or privilege is claimed are defined as follows:

1. A method for providing condom or prophylactic fitting and sizing based on girth measurement, recorded on computer-readable medium and capable of execution by a computer, the method comprising the steps of:
providing a tabular size guide/chart;
the size guide/chart correlates a range of girths to an appropriate condom size;
determining girth by direct measurement of an erect penis; and
correlating the girth measurement to an appropriate condom size.

2. The method of claim 1, further comprising the steps of
applying a prespecified girth tolerance for finding a size match to the corresponding girth with the appropriate condom size/systemic suggestion from the tabular size chart.

3. The method of claim 2, further comprising the steps of
displaying or providing for download a size chart correlating a series of prophylactic sizes to their corresponding range of penile girths;
displaying the series of prophylactic sizes to their corresponding range of penile girths;
providing a point-of-sale system;
accepting an order; and
accepting remittance of the correlated order information to the point of sale system using aforementioned computer or portable terminal is contemplated.

4. The method of claim 2, wherein
the sizing method is enabled by the software application running on a computer hardware platform of the present invention, which contains penile girth data;
a first step is for the user is to access the user interface using a computer or portable terminal;
from the terminal, the User proceeds to take their pre-measured penile girth and correlate it to one or multiple prophylactic sizes on a size chart to make or order and remit the correlated order information to the point of sale; and
the system will process the point of sale order and deliver the ordered product to a remote location.

5. The method of claim 2, wherein
the condom size chart allows users to correlate their penis girth to one of the condom sizes offered for sale;
the basis for helping the user find the right condom size based solely on their girth is the slippage-breakage risk algorithm.

6. The method of claim 5, wherein
the condom circumference is the first variable that is determined in the algorithm;
the condom circumference is measured in millimeters and is found by multiplying a condom's nominal width (provided by the manufacturer) by 2;
the second variable, “alpha” (a), represents the risk the user is exposed to for breaking or slipping out of a condom;
this risk is a ratio between the condom circumference (not nominal width) and penis girth, which should be between two prespecified endpoints by the inventor, an upper and lower bound;
alpha is represented as a range of percentages converted to decimals between two prespecified endpoints by the inventor, an upper and lower bound, with a prespecified step; and
the third variable is penis girth;
penis girth is a measurement that the user inputs themselves and is the circumference of an erect penis around the thickest part of the shaft.

7. The method of claim 6, wherein the breakage risk algorithm formulas include:
Condom Circumference [mm]=Nominal Width [mm]×2;
Suggested Nominal Width [mm]=(Penis Girth [mm]×α)÷2 ;
Suggested Nominal Width [mm]=(Penis Girth [in]×25.4×α)÷2;
Suggest Penis Girth [mm]=Condom Circumference [mm]÷α; and
Suggest Penis Girth [in]=(Condom Circumference [mm]×0.039370)÷α.

8. The method of claim 7, wherein
a size chart is produced as a result of the inputs in the algorithm and represents the recommended penis girths in relation to the risk variable for a given condom nominal width; and
a range of penis girths with an alpha that is between an additional two prespecified endpoints by the inventor, an upper and lower bound, within the initial prespecified endpoints to mitigate the risk of condom failure for the user.

9. The method of claim 8, wherein
the size chart represents the risk associated between a given penis girth and condom nominal width, there is a bit of overlap; and
using an alpha range between an additional two prespecified endpoints by the inventor, an upper and lower bound, within the initial prespecified endpoints to suggest a range of penis girths.

10. The method of claim 9, wherein
there is a prespecified penis girth tolerance as a result of the sizing and risk overlap mentioned above.

11. The method of claim 10, further comprising the step of
suggesting a user try a plurality of condom sizes with nominal widths both above and below the measured width to see which one fits best.

12. The method of claim 5, further comprising the steps of
only using penis girth to correlate it to a condom nominal width; and
using a tolerance factor to consider when choosing a condom nominal width size based on penis girth.

13. The method of claim 5, further comprising the steps of
creating a sizing chart to only use penis girth to correlate it to a condom nominal width; and
utilizing a tolerance factor to consider when choosing a condom nominal width based on penis girth.

14. The method of claim 5, further comprising the steps of
creating a sizing chart to only use penis girth to correlate it to a condom size; and
utilizing a tolerance factor to consider when choosing a condom size based on penis girth.

15. The method of claim 2, wherein
the condom size chart allows users to correlate their penis girth to one of the condom sizes offered for sale by the system;
the basis for helping the user find the right condom size based solely on their girth is the slippage-breakage risk algorithm.

16. A method for providing condom or prophylactic fitting and sizing based solely on a uni-dimensional girth measurement, comprising the steps of:
providing a size guide/chart;
the size guide/chart correlates a range of girths to an appropriate condom size;
determining girth by direct measurement of an erect penis; and
correlating the girth measurement to an appropriate condom size.

17. The method of claim 16, wherein
the size chart represents the risk associated between a given penis girth and condom nominal width, there is a bit of overlap.

18. The method of claim 17, wherein
utilizing a tolerance factor to consider when choosing a condom nominal width size based on penis girth; and
there is a pre-specified penis girth tolerance.

19. The method of claim 18, further comprising the step of
suggesting a user try a plurality of condom sizes with nominal widths both above and below the measured width to see which one fits best.

20. The method of claim 16, further comprising the steps of
only using penis girth to correlate it to a condom nominal width; and
using a tolerance factor to consider when choosing a condom nominal width size based on penis girth.
